(19) United States
(12) Patent Application Publication
(10) Pub. No.: US 2008/0317704 A1
(43) Pub. Date: Dec. 25, 2008

(54) Control of ice-crystal growth by non-proteinaceous substance

(76) Inventors: Hitoshi Obata, Amagasaki-shi (JP); Hidehisa Kawahara, Hirakata-shi (JP); Takashi Tomita, Toyonaka-shi (JP)

Correspondence Address: WENDEROTH, LIND & PONACK, L.L.P., 2033 K STREET N. W., SUITE 800, WASHINGTON, DC, 20006-1021, US

(21) Appl. No.: 12/222,863

(22) Filed: Aug. 18, 2008

Related U.S. Application Data
(63) Continuation of application No. 10/576,293, filed on Apr. 19, 2006.

(30) Foreign Application Priority Data
Oct. 22, 2003 (JP) 2003-362427

(51) Int. Cl. C08G 73/10; C12H 1/00; A01N 1/02; A61K 31/785; A01N 1/00

(52) U.S. Cl. 424/78.37; 528/422; 426/442; 47/2; 435/1.1

(57) ABSTRACT

The present invention makes it possible to attain various applications using antifreeze activity without an antifreeze protein. The agent for the inhibition of ice-crystal growth includes a non-proteinaceous substance, wherein an aqueous solution of the non-proteinaceous substance in a concentration of 10 mg/ml causes the deposition of non-flat disk-shaped ice crystals. The agent for the lowering of an ice-crystal growth initiation temperature includes a non-proteinaceous substance, wherein an aqueous solution of the non-proteinaceous substance in a concentration of 10 mg/ml shows thermal hysteresis by a temperature of 0.020° C. or higher. The agent for the control of water freezing includes a non-proteinaceous substance, wherein an aqueous solution of the non-proteinaceous substance in a concentration of 10 mg/ml shows thermal hysteresis by a temperature of 0.020° C. or higher and causes the deposition of non-flat disk-shaped ice crystals. The above non-proteinaceous substances are usually polymers each having a carbon chain as the main chain.

### TECHNICAL FIELD

The present invention relates to the control of ice-crystal growth by the use of a non-proteinaceous substance, and more particularly, to an agent for the inhibition of ice-crystal growth, an agent for the lowering of an ice-crystal growth initiation temperature, and an agent for the control of water freezing.

### BACKGROUND ART

The antifreeze activity of an antifreeze protein is briefly described in the article “Synthesis of Anti-freeze Sugar Proteins; Exploring the Mystery of Anti-freeze Fishes” by Shin-ichiro Nishimura in Gendai Kagaku (Modern Chemistry), published by Tokyo Kagaku Dojin, April 1999, Vol. 337, pp. 56-62. The antifreeze protein (abbreviated sometimes as AFP) is a special kind of protein found in fishes, insects, plants, and other organisms living in polar areas. For example, the body fluid of ordinary fishes freezes at around −0.8° C., while the body fluid of fishes having an AFP in their bodies is characterized in that it does not freeze when the temperature is lowered even below −2° C. Because seawater freezes at about −1.9° C., fishes having an AFP in their bodies can live without suffering the freezing of their body fluids.

In general, the lowering of freezing point is often explained by the rule of the molar lowering of freezing point that the degree of freezing point lowering is in direct proportion to the molar concentration of a solute. However, the degree of freezing point lowering is not in direct proportion to the molar concentration of an AFP. In other words, the AFP prevents the freezing of a body fluid by a mechanism different from the rule of the molar lowering of freezing point, that is, by being adsorbed specifically to the growth surface of an ice crystal grown in a living body, thereby inhibiting the growth of the ice crystal.

The growth of an ice crystal will hereinafter be concretely described with reference to FIGS. 1, 2, and 3. FIG. 1 is a schematic view showing the growth of an ice crystal in the absence of an AFP. FIGS. 2 and 3 are schematic views showing two examples of the growth of an ice crystal in the presence of an AFP. As shown in FIG. 1, when the minimum nucleus of ice is formed, this minimum nucleus is usually grown both in the direction of a-axis and in the direction of c-axis. In this case, the growth rate is about 100 times higher in the direction of a-axis than in the direction of c-axis; therefore, a disk-shaped nucleus of ice (i.e., ice crystal) 1 is formed. In contrast, as shown in FIG. 2, when an AFP is present, as soon as the minimum nucleus of ice is formed, the AFP is attached or adsorbed to the surface of the nucleus in the direction of a-axis (i.e., prism surface), thereby preventing the growth of an ice crystal in the direction of a-axis, so that a hexagonal ice crystal 2 is formed. The hexagonal ice crystal 2 is grown as shown in FIG. 3 by stacking small hexagonal columns in the direction of c-axis to become a bipyramidal ice crystal 3. The AFP may sometimes prevent the growth of an ice crystal in the direction of c-axis, in which case the ice crystal remains in hexagonal shape (see FIG. 2). In any case, the presence of an AFP causes the change of ice crystals to a shape different from the ordinary case (i.e., flat disk shape).

The antifreeze activity of AFPs is characterized not only by the change of ice crystal in shape as described above but also by the fact that ice crystals are inhibited from uniting with each other because the crystal growth is prevented, for example, in the direction of c-axis. It can therefore be said that the prevention of growth of bulky ice crystals is involved in antifreeze activity.

Furthermore, it is also involved in antifreeze activity to show thermal hysteresis. When an aqueous solution containing an AFP dissolved therein is cooled excessively for complete freezing once and the temperature of this system is then raised gradually, melting begins. When the temperature is lowered slightly below the melting temperature (i.e., melting point) and the system is then left undisturbed for a longtime, freezing begins in ordinary cases (i.e., the freezing point corresponds with the melting point). However, when an AFP is present, freezing does not begin until the temperature is further lowered. The difference between the above melting temperature (i.e., melting point) and the refreezing temperature (i.e., freezing point) is referred to as thermal hysteresis. It is one of the requirements for antifreeze activity that the system shows thermal hysteresis.

Thus the antifreeze activity means that 1) the shape of ice crystals is changed (to a shape different from the flat disk shape; herein after the term “non-flat disk shape” or “non-flat disk-shaped” is used to express a shape different from the flat disk shape); 2) ice crystals are inhibited from uniting with each other; and 3) the system shows thermal hysteresis. Of these requirements, the first, i.e., 1) the shape of ice crystals is changed, and the second, i.e., 2) ice crystals are inhibited from uniting with each other, are both based on the prevention of ice-crystal growth and can therefore be equated with each other. In this context, the antifreeze activity can be found, if 1) the shape of ice crystals is changed (to a non-flat disk shape) and 2) the system shows thermal hysteresis.

The AFPs having such antifreeze activity have been studied to develop various applications. For example, the following applications have been intensively developed: improvements in quality or texture of frozen food (see, e.g., WO 96/39878, WO 96/11586, WO 98/4699, WO 98/4147, WO 98/4148, JP-A 2000-157195, WO 99/37164, WO 99/37673, WO 00/53025, WO 00/53026, WO 00/53027, WO 00/53028, WO 00/53029, and WO 99/37673); improvements in the freezing resistance of living tissues or body fluids (see, e.g., WO 91/10361, WO 97/36547, and WO 00/00512); and ice thermal storage systems (see, e.g., JP-A 8-75328).

However, antifreeze proteins are very expensive and cost 1,000,000 yen per gram at the present time. Furthermore, antifreeze proteins are easily denatured by heat and may cause antigen-antibody reaction when applied to living bodies. There has so far been found no non-proteinaceous substance having antifreeze activity.

Japanese Patent No. 3111219 discloses a cryogenic transport system using polyvinyl alcohol, and in the section of Examples, it is shown that the use of polyvinyl alcohol prevents the recrystallization of granular ice crystals. However, this reference is completely silent on what behavior polyvinyl alcohol shows about thermal hysteresis as an important requirement for antifreeze activity.

### DISCLOSURE OF INVENTION

The present invention has been completed under the above circumstances, and it is an object of the present invention to attain the development of various applications using antifreeze activity without an antifreeze protein.

The present inventors have intensively studied to solve the above problems, and as a result, they have first found that there are non-proteinaceous substances having antifreeze activity (e.g., acrylamide homopolymers) and demonstrated that an aqueous solution of each of these substances in a concentration of 10 mg/ml causes the deposition of non-flat disk-shaped ice crystals and shows thermal hysteresis by a temperature of 0.020° C. or higher, thereby completing the present invention. The non-proteinaceous substances can be used for various applications in place of antifreeze proteins (AFPs).

Thus the present invention provides an agent for the inhibition of ice-crystal growth, comprising a non-proteinaceous substance, wherein an aqueous solution of the non-proteinaceous substance in a concentration of 10 mg/ml causes the deposition of non-flat disk-shaped ice crystals. The agent for the inhibition of ice-crystal growth can be added to a heat medium in an ice thermal storage system or can also be added to frozen food.

The present invention further provides an agent for the lowering of an ice-crystal growth initiation temperature, comprising a non-proteinaceous substance, wherein an aqueous solution of the non-proteinaceous substance in a concentration of 10 mg/ml shows thermal hysteresis by a temperature of 0.020° C. or higher. The agent for the lowering of an ice-crystal growth initiation temperature can be sprayed or applied onto a portion for the possible attachment of an ice crystal to prevent the attachment of an ice crystal. It can also be sprayed or applied onto a ground surface or an agricultural crop to prevent the freezing or frost damage thereof.

The present invention further provides an agent for the control of water freezing, comprising a non-pro-teinaceous substance, wherein an aqueous solution of the non-proteinaceous substance in a concentration of 10 mg/ml shows thermal hysteresis by a temperature of 0.020° C. or higher and causes the deposition of non-flat disk-shaped ice crystals. The agent for the control of water freezing can be injected into a living tissue or a body fluid to prevent the damage of the living tissue or the freezing of the body fluid under a freezing point thereof (i.e., at a sub-zero temperature).

The above non-proteinaceous substances are, for example, polymers each having a carbon chain as the main chain.

The non-proteinaceous substances of the present invention can find various applications using anti-freeze activity without an antifreeze protein because an aqueous solution of each of the non-proteinaceous substances in a concentration of 10 mg/ml causes the deposition of non-flat disk-shaped ice crystals and shows thermal hysteresis by a temperature of 0.020° C. or higher.

The inhibition of ice-crystal growth, the lowering of an ice-crystal growth initiation temperature, and the control of water freezing may fall within the concept “control of ice-crystal growth.” It can therefore be said that the present invention provides an agent for the control of ice-crystal growth, comprising anon-proteinaceous substance, which is, for example, a polymer having a carbon chain as the main chain, wherein an aqueous solution of the non-proteinaceous substance shows thermal hysteresis by a temperature of 0.020° C. or higher and/or causes the deposition of non-flat disk-shaped ice crystals.

### BRIEF DESCRIPTION OF DRAWINGS

FIG. 1 is a schematic view for the explanation of an ice crystal deposited from an aqueous solution of a substance having no antifreeze activity.

FIG. 2 is a schematic view for the explanation of an ice crystal deposited from an aqueous solution of a substance having antifreeze activity.

FIG. 3 is a schematic view for the explanation of another ice crystal deposited from an aqueous solution of a substance having antifreeze activity.

FIG. 4 is a photograph under a microscope showing an ice crystal in an aqueous solution of polyacrylamide.

FIG. 5 is a photograph under a microscope showing an ice crystal in an aqueous solution of polyvinyl alcohol.

FIG. 6 is a photograph under a microscope showing an ice crystal in an aqueous solution of an acryloyl pyrrolidine homopolymer.

FIG. 7 is a photograph under a microscope showing an ice crystal in an aqueous solution of an n-butyl vinyl ether maleic anhydride copolymer.

FIG. 8 is a photograph under a microscope showing an ice crystal in an aqueous solution of an isobutyl vinyl ether maleic anhydride copolymer.

### BEST MODE FOR CARRYING OUT THE INVENTION

As described above, the present invention relates to non-proteinaceous substances having antifreeze activity. An aqueous solution of each of the non-proteinaceous substances in a concentration of 10 mg/ml causes the change of ice crystals in shape (i.e., the deposition of non-flat disk-shaped ice crystals) and shows thermal hysteresis.

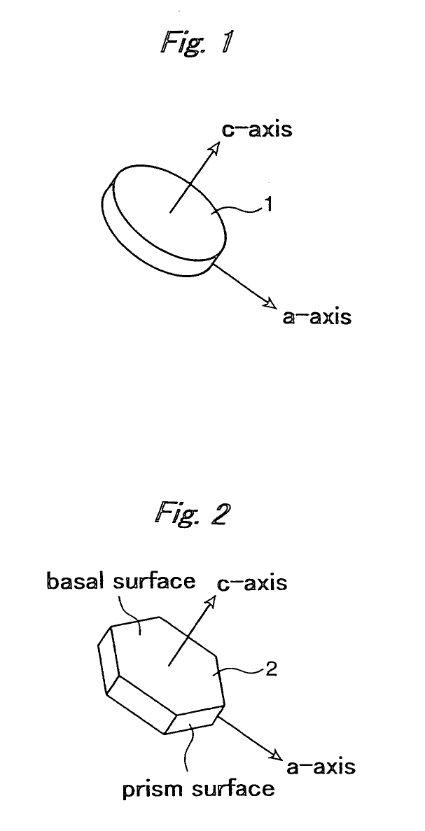

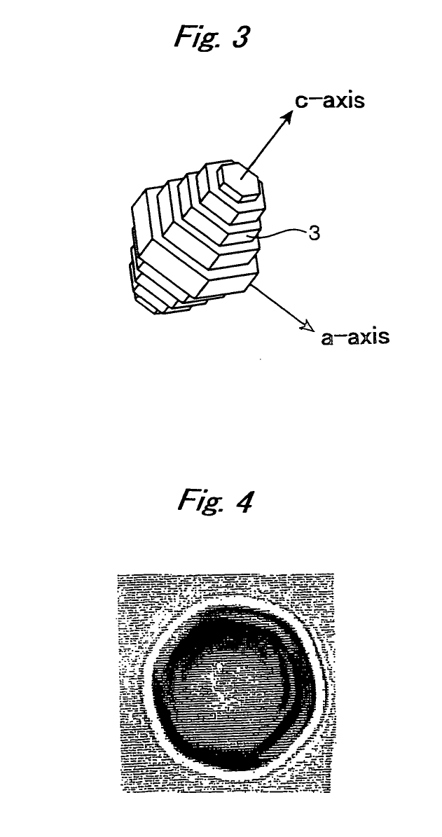

The change of ice crystals in shape means that when an aqueous solution of one of the non-proteinaceous substances of the present invention in a concentration of 10 mg/ml is cooled, the ice crystals are not grown in such a shape as the flat disk-shaped ice crystal 1 shown in FIG. 1, but grown, for example, in such a shape as the hexagonal (i.e., flat hexagonal columnar) ice crystal 2 shown in FIG. 2 or the bipyramidal ice crystal 3 shown in FIG. 3. The change of ice crystals in shape in this manner is attributed to the inhibition of ice-crystal growth in the particular direction or directions (e.g., in the direction of a-axis (in the direction perpendicular to the prism surface) or both in the direction of such a-axis and in the direction of c-axis (in the direction perpendicular to the basal surface)).

The thermal hysteresis is a temperature defined as follows. An aqueous solution of an object substance in a concentration of 10 mg/ml is prepared. The aqueous solution is cooled excessively for complete freezing once, and then the temperature of this system is raised gradually to cause the melting of ice crystals. At such a stage that a slight amount of ice crystals remain (e.g., at such a stage that only one ice crystal having a size of about 0.08 to 0.1 mm remains in the field of view having an area of 0.1 mm×0.1 mm), the system begins to be cooled gradually (e.g., at a cooling rate of about 1° C./min.) again, so that the ice crystals are grown again. The temperature at which a slight amount of ice crystals remain (i.e., melting point) and the temperature at which the regrowth of ice crystals begins to be observed (i.e., freezing point) are measured to determine the difference between these temperatures (i.e., melting point minus freezing point). The above difference in temperature (i.e., melting point minus freezing point) will inevitably contains an error of measurement. However, when the above difference in temperature reaches 0.020° C. or higher, it can be said that thermal hysteresis is found, even taking such an error of measurement into consideration. Therefore, the non-proteinaceous substances of the present invention are substances showing the above difference in temperature (i.e., melting point minus freezing point) of 0.020° C. or higher.

Examples of the non-proteinaceous substances having antifreeze activity (e.g., the property of causing the change of ice crystals in shape and the property of showing thermal hysteresis) as described above may include, for example, polymers each having a carbon chain as the main chain (e.g., acrylamide homopolyemrs).

The non-proteinaceous substances having anti-freeze activity can find various applications depending upon the contents of the above antifreeze activity. For example, the property of causing the change of ice crystals in shape can be utilized in an agent for the inhibition of ice-crystal growth. In this case, ice crystals can be inhibited from uniting with each other by the inhibition of ice-crystal growth. The agent for the inhibition of ice-crystal growth may be added, for example, to a heating medium in an ice thermal storage system or may also be added to frozen food (e.g., ice cream). The use of the above agent in an ice thermal storage system makes it possible to prevent an operation trouble of the system, which is caused by ice crystals uniting with each other when deposited from the heating medium. In this regard, however, because the amount of ice crystals themselves is not substantially changed, there is no possibility that the efficiency of thermal storage in the system would be reduced. In addition, the non-proteinaceous substances of the present invention have an effect even in a relatively small amount and thus cause no excessive overcooling. Furthermore, the non-proteinaceous substances of the present invention are chemically stable and thus make it possible to operate the system for a long time while maintaining the efficiency of operation. When added to frozen food, the non-proteinaceous substances of the present invention make it possible to prevent deterioration in the texture of food, which is caused by ice crystals uniting with each other in the food.

On the other hand, the property of showing thermal hysteresis, which belongs to the non-proteinaceous sub-stances of the present invention, can be utilized in an agent for the lowering of an ice-crystal growth initiation temperature. The agent for the lowering of an ice-crystal growth initiation temperature may be sprayed or applied onto a portion for possible attachment of ice crystals (e.g., aircraft wings, electric cables) to prevent the attachment of ice crystals or may also be sprayed or applied onto a ground surface (e.g., road surface, soil surface) or an agricultural crop to prevent the freezing or frost damage thereof. The non-proteinaceous substances of the present invention, particularly polyacrylamide, are characterized in that they are less corrosive to metals and they have a small environmental load because they are effective even when added in a small amount.

When the property of inhibiting the growth of ice crystals and the property of showing thermal hysteresis are both utilized, the non-proteinaceous substances of the present invention can be used in an agent for the control of water freezing. The agent for the control of water freezing can be injected, for example, into a living tissue or a body fluid to prevent the damage of the living tissue or the freezing of the body fluid under a freezing point thereof. More specifically, they can be utilized for an improvement in the freezing resistance of hatchery fishes, the frozen storage of sperms or organs, cryosurgery, and other applications. The substances of the present invention are non-proteinaceous and thus, for example, there is no possibility that they cause antigen-antibody reaction even when used for the storage of organs.

### EXAMPLE

The present invention will hereinafter be further illustrated by an example; however, the present invention is not limited to this example and can be carried out after appropriate modifications are made within a range in conformity to the purport of the foregoing and following descriptions, all of these modified ones falling within the technical scope of the present invention.

In the example of experiment described below, the following substances were examined for their antifreeze activity.

1) Polyacrylamide: available from Aldrich Co.; product number “43494-9” (Aldrich General Catalog 2003-2004); weight average molecular weight (the value in the catalog), 10,000

2) Polyvinyl alcohol: available from Kuraray Co., Ltd.; trade name “Kuraray PVA-205” (“Kuraray Poval” in the catalog of Kuraray Co., Ltd.); saponification value (the value in the catalog), 88.0±1.5 mole %; viscosity (4%, 20° C.; the value in the catalog), 5.0±0.4 mPa·s

3) Acryloyl pyrrolidine homopolymer: number average molecular weight, 1,200

4) n-Butyl vinyl ether maleic anhydride copolymer: completely neutralized form

This n-butyl vinyl ether maleic anhydride copolymer was obtained as follows.

A 300-mL flask with a stirrer, a thermometer, a reflux condenser, a nitrogen gas introduction tube, and a dropping funnel was charged with 15 g of maleic anhydride, 69.2 g of methyl t-butyl ether (MTBE), and 0.3 g (1.0 wt % relative to the copolymer produced) of polyisobutylvinylether (Lutonal I-60, available form BASF Aktiengesellschaft, K value=60) as a flocculation inhibitor, followed by heating with stirring until the temperature reached 55° C., so that the maleic anhydride was dissolved. On the other hand, the dropping funnel was charged with 16.8 g (1.1 times greater moles relative to the maleic anhydride) of n-butyl vinyl ether and 75 mg (0.5 wt % relative to the maleic anhydride) of 2,2′-azobis(2,4-dimethylvaleronitrile) [V-65] as a polymerization initiator. The air in the flask and the air in the dropping funnel were replaced with nitrogen gas over 10 minutes.

While the temperature in the flask was kept at 55° C., the content of the dropping funnel was dropped into the flask over 1 hour. After completion of dropping, stirring was continued at a temperature of 55° C. to 60° C. for another two hours. The polymerization suspension had a copolymer concentration of 30% by weight.

A portion of the polymerization suspension after completion of the reaction was taken out and analyzed by LC (liquid chromatography). As a result, no maleic anhydride was detected, making sure that the reaction had been completed. The above polymerization suspension was easily taken out of the flask, and there was found neither flocculation nor adhesion of the copolymer to the blades of the stirrer or to the internal wall of the flask. The polymerization suspension obtained was filtered by suction using filter paper of 1 μm in pore diameter, at which time the copolymer was easily separated and the filtrate was clear.

The copolymer was dried at a temperature of 60° C. under reduced pressure to give 30.4 g of white powder. The resulting copolymer had a weight average molecular weight of 31,000 and an average particle diameter of 7.8 μm, and the accumulative rate (under sieving) of particles having a diameter of 1 μm or smaller was 0%.

The white powder thus obtained was added to an aqueous solution of sodium hydroxide (containing an equivalent amount of sodium hydroxide relative to carboxyl groups), followed by stirring at room temperature, to give a pale yellow uniform aqueous solution.

5) Isobutyl vinyl ether maleic anhydride copolymer: completely neutralized form

In the same manner as described in the case of 5) n-butyl vinyl ether maleic anhydride copolymer, except that isobutyl vinyl ether was used in place of n-butyl vinyl ether, white powder having a weight average molecular weight of 30,000 was obtained. The white powder obtained was hydrolyzed in the same manner as described in the case of 5) n-butyl vinyl ether maleic anhydride copolymer to give a pale yellow uniform aqueous solution.

### Example of Experiment

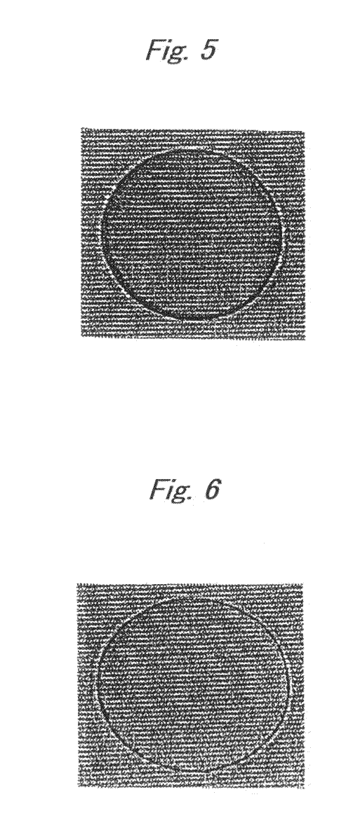

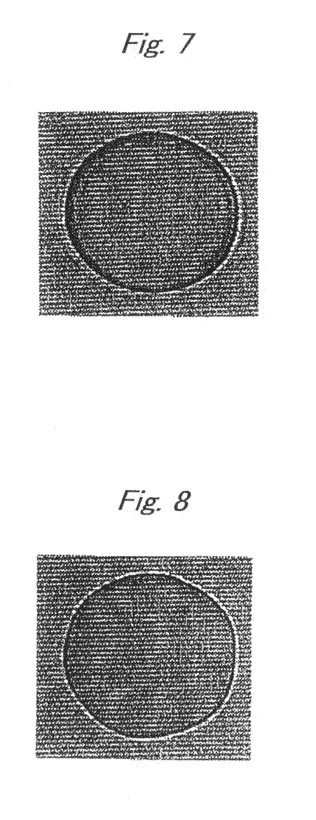

Aqueous solutions of the above substances (in a concentration of 10 mg/ml) were prepared separately. Each of these aqueous solutions was placed on a freezing stage with a temperature controller (LK-600PM, available from Linkam Scientific Instruments Ltd.) and cooled to −30° C. for complete freezing. While an observation was made with a phase-contrast microscope (at a magnification of 100 times), the temperature was raised (at a temperature rising rate of 100° C./min.) to about −1° C. until only one ice single crystal having a size of about 0.08 to 0.1 mm remained. The field of view was changed to a size of 0.1 mm×0.1 mm (i.e., the image of the ice single crystal was enlarged in the frame). In this condition, the ice crystal was gradually cooled (at a cooling rate of 1° C./min.), the growth of which was observed. The difference (i.e., thermal hysteresis) between the temperature at which only one single crystal remained (i.e., melting point) and the temperature at which the growth of the ice crystal was observed (i.e., freezing point) was determined. Furthermore, the shape of the ice crystal grown by the above operation was observed. The results are shown in Table 1 and FIGS. 4 to 8. FIG. 4 shows an ice crystal in the aqueous solution of the polyacrylamide; FIG. 5, an ice crystal in the aqueous solution of the polyvinyl alcohol; FIG. 6, an ice crystal in the aqueous solution of the acryloyl pyrrolidine homopolymer; FIG. 7, an ice crystal in the aqueous solution of the n-butyl vinyl ether maleic anhydride copolymer; and FIG. 8, an ice crystal in the aqueous solution of the isobutyl vinyl ether maleic anhydride copolymer.

TABLE 1

| | Difference between | |
|---|---|---|
| | melting point and | |
| | freezing point | pH of |
| | (° C.) | aqueous solution |
| Polyacrylamide | 0.021 | 5.5 |
| Polyvinyl alcohol | 0.016 | 6.0 |
| Acryloyl pyrrolidine | 0 | 4.5 |
| homopolymer | | |
| n-Butyl vinyl ether | 0.0014 | 11.0 |
| maleic anhydride | | |
| copolymer | | |
| Isobutyl vinyl ether | 0.00125 | 10.7 |
| maleic anhydride | | |
| copolymer | | |

As can be seen from Table 1 and FIGS. 5 to 8, the respective aqueous solutions of the polyvinyl alcohol, the acryloyl pyrrolidine homopolymer, the n-butyl vinyl maleic anhydride copolymer, or the isobutyl vinyl ether maleic anhydride copolymer caused the deposition of flat disk-shaped ice crystals by making no change of ice crystals in shape and showed substantially no thermal hysteresis.

In contrast to these results, as can be seen from Table 1 and FIG. 4, the aqueous solution of the polyacrylamide caused the deposition of bipyramidal ice crystals by making the change of ice crystals in shape and showed thermal hysteresis.

### INDUSTRIAL APPLICABILITY

The non-proteinaceous substances of the present invention can find various applications using anti-freeze activity without an antifreeze protein because an aqueous solution of each of the non-proteinaceous substances in a concentration of 10 mg/ml causes the deposition of non-flat disk-shaped ice crystals and shows thermal hysteresis by a temperature of 0.020° C. or higher.

1-11. (canceled)

12. A method for inhibiting ice-crystal growth in a material, which comprises adding to the material a non-proteinaceous substance, wherein an aqueous solution of the non-proteinaceous substance in a concentration of 10 mg/ml causes deposition of a non-flat disk-shaped ice crystal, and wherein the non-proteinaceous substance is a polyacrylamide.

13. The method according to claim 12, wherein the material is a heat medium in an ice thermal storage system.

14. The method according to claim 12, wherein the material is food to be frozen.

15. A method for lowering an ice-crystal growth initiation temperature in a material, which comprises adding to the material a non-proteinaceous substance, wherein an aqueous solution of the non-proteinaceous substance in a concentration of 10 mg/ml shows thermal hysteresis by a temperature of 0.020° C. or higher, and wherein the non-proteinaceous substance is a polyacrylamide.

16. The method according to claim 15, wherein the non-proteinaceous substance is sprayed or applied onto a material susceptible to attachment of an ice crystal to prevent the attachment of an ice crystal.

17. The method according to claim 15, wherein the non-proteinaceous substance is sprayed or applied onto a ground surface or an agricultural crop to prevent freezing or frost damage thereof.

18. A method for controlling water freezing, which comprises adding to a water-containing material a non-proteinaceous substance, wherein an aqueous solution of the non-proteinaceous substance in a concentration of 10 mg/ml shows thermal hysteresis by a temperature of 0.020° C. or higher and causes deposition of a non-flat disk-shaped ice crystal, and wherein the non-proteinaceous substance is a polyacrylamide.

19. The method according to claim 18, wherein the non-proteinaceous substance is injected into a living tissue or a body fluid to prevent damage of the living tissue or freezing of the body fluid under a freezing point thereof.
